(19) United States
(12) Patent Application Publication
(10) Pub. No.: US 2006/0120987 A1
(43) Pub. Date: Jun. 8, 2006

(54) Skin-friendly detergent mixtures

(76) Inventors: Claudia Panzer, Grevenbroich (DE); Manfred Weuthen, Langenfeld (DE); Rolf Wachter, Duesseldorf (DE)

Correspondence Address: COGNIS CORPORATION;PATENT DEPARTMENT, 300 BROOKSIDE AVENUE, AMBLER, PA, 19002, US

(21) Appl. No.: 11/271,187

(22) Filed: Nov. 10, 2005

(30) Foreign Application Priority Data
Nov. 12, 2004 (DE) 102004054843.9
Mar. 22, 2005 (DE) 102005013132.8

(51) Int. Cl. A61K 8/73; C11D 3/384

(52) U.S. Cl. 424/70.13; 510/234

(57) ABSTRACT

The invention relates to the use of film-forming cationic biopolymers for improving the dermatological compatibility of manual dishwashing detergents. Chitosan or chitosan derivatives are preferably used for this purpose, preferably in the absence of anionic surfactants.

### CROSS REFERENCE TO RELATED APPLICATIONS

This application claims priority under 35 U.S.C. §119 from German Patent Applications No. 102004054843.9, filed Nov. 12, 2004 and No. 102005013132.8, filed Mar. 22, 2005.

### BACKGROUND OF THE INVENTION

Liquid or gel-form detergent mixtures are normally used for the manual cleaning of hard surfaces, particularly dishes. These liquids/gels should be highly concentrated, have a low cold cloud point, generate sufficient foam, despite high levels of fats in the wash liquor, clean efficiently and should not irritate the skin.

Solutions for this requirement profile are known from the prior art. U.S. Pat. No. 6,013,616, the entire contents of which are incorporated herein by reference, discloses skin-friendly surfactant mixtures which contain monoglycerol ether sulfates in combination with a mixture of condensation products of fatty acid isethionates, taurides or sarcosinates and which are suitable for the production of, or for use as, dishwashing detergents. The use of chitosan as an optional film-forming additive, among others, is mentioned. WO 99/03959 A1 describes detergent mixtures which contain so-called esterquats, chitosan or chitosan derivatives and protein hydrolyzates alongside one another. However, the effect of the chitosan in the ternary mixture is not actually disclosed.

However, there is a constant need to improve surfactant mixtures, more particularly those for manual dishwashing, in their effect on the human skin, more particularly in their dermatological compatibility. More particularly, it ought to be possible to obtain an improvement in the dermatological properties, irrespective of the composition of the dishwashing detergent.

### BRIEF SUMMARY OF THE INVENTION

It has been found that the use of certain cationic film-forming biopolymers can solve the problem stated above.

The present invention thus relates firstly to the use of chitosan and/or chitosan derivatives for improving the dermatological compatibility of detergent mixtures. The aqueous preparations of the invention may preferably contain surfactants, which can be nonionic, cationic and/or amphoteric surfactants being suitable. Preparations free from anionic surfactants are particularly preferred.

### DETAILED DESCRIPTION OF THE INVENTION

In the context of the present teaching, dermatological compatibility is determined by a patch test. In this test, plasters containing the test substances (FineChambers®) are applied to the backs of 20 volunteers and left there for 24 hours. A highly dilute aqueous solution of the particular test formulation (concentration 1 to 2% by weight active substance for example) is used for the test. Following removal of the plasters, the skin of the volunteers is visually examined after 6, 24, 48 and 78 hours. The features skin reddening, oedema formation, exfoliation and cracking are observed.

Chitosans are biopolymers known per se which belong to the group of hydrocolloids. Chemically, they are partly deacetylated chitins differing in their molecular weights which contain the following—idealized—monomer unit: 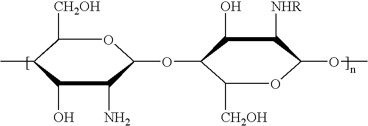
In contrast to most hydrocolloids, which are negatively charged at biological pH values, chitosans are cationic biopolymers under these conditions. The positively charged chitosans are capable of interacting with oppositely charged surfaces and are therefore used in cosmetic hair-care and body-care products and pharmaceutical preparations. Chitosans are produced from chitin, preferably from the shell residues of crustaceans which are available in large quantities as inexpensive raw materials. In a process described for the first time by Hackmann et al., the chitin is normally first deproteinized by addition of bases, demineralized by addition of mineral acids and, finally, deacetylated by addition of strong bases, the molecular weights being distributed over a broad spectrum. The average molecular weight is preferably in the range from 100 to 5,000,000 g/mol and more particularly in the range from 800,000 to 1,200,000 g/mol. The chitosans or their derivatives preferably have molecular weights of 50,000 to 1,200,000 g/mol. Such chitosans preferably have a Brookfield viscosity (1% by weight in glycolic acid) below 5,000 mPas, a degree of deacetylation of up to 100%, preferably up to 99% and more particularly in the range from 80 to 88% and, preferably, an ash content of less than 0.3% by weight. Besides the chitosans as typical cationic biopolymers, anionically or nonionically derivatized chitosans, for example carboxylation, succinylation or alkoxylation products, may also be used for the purposes of the invention, although chitosan is preferred to its derivatives. In the context of the present technical teaching, preferred chitosans or chitosan derivatives are those which have a molecular weight above 50,000 g/mol and, more particularly, above 100,000 g/mol. Other preferred molecular weight ranges are 50,000 to 1,000,000 and 50,000 to 300,000. However, chitosans with molecular weights in the range from 500,000 to 5,000,000 g/mol and those with molecular weights in the range from 300,000 to 2,000,000 g/mol are particularly preferred. Besides the chitosans as typical cationic biopolymers, anionically or nonionically derivatized chitosans, for example carboxylation, succinylation or alkoxylation products, are also suitable for the purposes of the invention.

Chitosan itself may be present as a solid powder or, preferably, as an aqueous solution. These solutions advantageously contain 0.01 to 5% by weight (active substance) of chitosan or chitosan derivative. Aqueous solutions containing 0.01 to 2.5% by weight, preferably 0.05 to 1.0% by weight and more particularly 0.05 to 0.5% by weight chitosan or chitosan derivative are preferred. These aqueous solutions may also contain organic and/or inorganic acids to adjust an acidic pH of advantageously <7 and, more particularly, in the range from 6.5 to 4.

The aqueous preparations may preferably contain other ingredients. Surfactants are particularly important in this regard, nonionic, cationic and/or amphoteric surfactants being suitable. Preparations free from anionic surfactants are particularly preferred because the chitosans are capable of reacting with the anionic functionalities. However, the present technical teaching also encompasses preparations where chitosan derivatives and anionic surfactants are formulated together.

In one preferred embodiment, however, the preparations are free from so-called esterquats. Esterquats are generally understood to be quaternized fatty acid triethanolamine ester salts, These are substances which correspond to formula (I), (II) or (III): 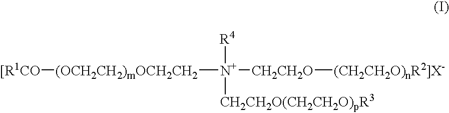
in which R¹CO is an acyl group containing 6 to 22 carbon atoms, R² and R³ independently of one another represent hydrogen or have the same meaning as R¹CO, R⁴ is an alkyl group containing 1 to 4 carbon atoms or a (CH₂CH₂O)_{q}H group, m, n and p together stand for 0 or numbers of 1 to 12, q is a number of 1 to 12 and X is halide, alkyl sulfate or alkyl phosphate. 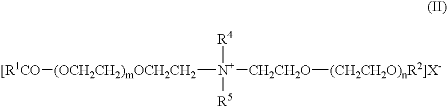
in which R¹CO is an acyl group containing 6 to 22 carbon atoms, R² is hydrogen or has the same meaning as R¹CO, R⁴ and R⁵ independently of one another are alkyl groups containing 1 to 4 carbon atoms, m and n together stand for 0 or numbers of 1 to 12 and X stands for halide, alkyl sulfate or alkyl phosphate. 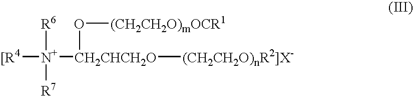
in which R¹CO is an acyl group containing 6 to 22 carbon atoms, R² is hydrogen or has the same meaning as R¹CO, R⁴, R⁶ and R⁷ independently of one another are alkyl groups containing 1 to 4 carbon atoms, m and n together stand for 0 or numbers of 1 to 12 and X stands for halide, alkyl sulfate or alkyl phosphate. The use of surfactants corresponding to formulae (I), (II) and (III) together with chitosans or derivatives thereof in the formulation disclosed in WO 99/03959 A1 is not the subject of the present technical teaching.

By contrast, it is preferred that the aqueous preparations according to the invention contain nonionic surfactants, preferably of the alkyl (oligo)glycoside (APG) type. Such compounds correspond to formula (IV):
R⁸O—[G]ₚ (IV)
in which R⁸ is an alkyl and/or alkenyl group containing 4 to 22 carbon atoms, G is a sugar unit containing 5 or 6 carbon atoms and p is a number of 1 to 10. They may be obtained by the relevant methods of preparative organic chemistry. The alkyl and/or alkenyl oligoglycosides may be derived from aldoses or ketoses containing 5 or 6 carbon atoms, preferably glucose. Accordingly, the preferred alkyl and/or alkenyl oligoglycosides are alkyl and/or alkenyl oligoglucosides. The index p in general formula (IV) indicates the degree of oligomerization (DP), i.e. the distribution of mono- and oligoglycosides, and is a number of 1 to 10. Whereas p in a given compound must always be an integer and, above all, may assume a value of 1 to 6, the value p for a certain alkyl oligoglycoside is an analytically determined calculated quantity which is generally a broken number. Alkyl and/or alkenyl oligoglycosides having an average degree of oligomerization p of 1.1 to 3.0 are preferably used. Alkyl and/or alkenyl oligoglycosides having a degree of oligomerization of less than 1.7 and, more particularly, between 1.2 and 1.4 are preferred from the applicational point of view. The alkyl or alkenyl radical R⁸ may be derived from primary alcohols containing 4 to 11 and preferably 8 to 10 carbon atoms. Typical examples are butanol, caproic alcohol, caprylic alcohol, capric alcohol and undecyl alcohol and the technical mixtures thereof obtained, for example, in the hydrogenation of technical fatty acid methyl esters or in the hydrogenation of aldehydes from Roelen's oxosynthesis. Alkyl oligoglucosides having a chain length of C₈ to C₁₀ (DP=1 to 3), which are obtained as first runnings in the separation of technical C₈₋₁₈ coconut oil fatty alcohol by distillation and which may contain less than 6% by weight of C₁₂ alcohol as an impurity, and also alkyl oligoglucosides based on technical C_{9/11} oxoalcohols (DP=1 to 3) are preferred. In addition, the alkyl or alkenyl radical R⁸ may also be derived from primary alcohols containing 12 to 22 and preferably 12 to 14 carbon atoms. Typical examples are lauryl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol and technical mixtures thereof which may be obtained as described above. Alkyl oligoglucosides based on hydrogenated C_{12/14} cocoalcohol with a DP of 1 to 3 are preferred.

Other preferred nonionic surfactants are fatty alcohols, fatty alcohol alkoxylates, more particularly ethoxylates, hydroxylated derivatives of fatty alcohols, alkoxylated, preferably ethoxylated, carboxylic acid and polyethylene glycols and derivatives thereof. Mixtures of these surfactants are also possible.

Another group of suitable and preferred surfactants are the betaines. Betaines are known surfactants which are mainly produced by carboxyalkylation, preferably carboxymethylation, of aminic compounds. The starting materials are preferably condensed with halocarboxylic acids or salts thereof, more particularly with sodium chloroacetate, 1 mol of salt being formed per mol of betaine. The addition of unsaturated carboxylic acids, for example acrylic acid, is also possible. Examples of suitable betaines are the carboxyalkylation products of secondary and, in particular, tertiary amines. Typical examples are the carboxymethylation products of hexyl methyl amine, hexyl dimethyl amine, octyl dimethyl amine, decyl dimethyl amine, dodecyl methyl amine, dodecyl dimethyl amine, dodecyl ethyl methyl amine, C_{12/14} cocoalkyl dimethyl amine, myristyl dimethyl amine, cetyl dimethyl amine, stearyl dimethyl amine, stearyl ethyl methyl amine, oleyl dimethyl amine, C_{16/18} tallow alkyl dimethyl amine and technical mixtures thereof. Other suitable betaines are carboxyalkylation products of amidoamines. Typical examples are reaction products of fatty acids containing 6 to 22 carbon atoms, namely caproic acid, caprylic acid, capric acid, lauric acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and technical mixtures thereof, with N,N-dimethyl aminoethyl amine, N,N-dimethyl aminopropyl amine, N,N-diethyl aminoethyl amine and N,N-diethyl aminopropyl amine which are condensed with sodium chloroacetate. It is preferred to use a condensation product of C_{8/18} cocofatty acid-N,N-dimethyl aminopropyl amide with sodium chloroacetate. Other suitable starting materials for the betaines to be used in accordance with the invention are imidazolines. Imidazolines are also known compounds which may be obtained, for example, by cyclizing condensation of 1 or 2 mol of fatty acid with polyfunctional amines, for example aminoethyl ethanolamine (AEEA) or diethylene triamine. The corresponding carboxyalkylation products are mixtures of different open-chain betaines. Typical examples are condensation products of the above-mentioned fatty acids with AEEA, preferably imidazolines based on lauric acid or—again—C_{12/14} cocofatty acid which are subsequently betainized with sodium chloroacetate.

The water-based preparations according to the invention may also preferably contain anionic surfactants. Suitable anionic surfactants are, for example, alkyl benzenesulfonates, alkyl sulfonates and alkyl ether sulfates. Alkyl benzenesulfonates preferably correspond to the formula R′—Ph—SO₃X, in which R′ is a branched, but preferably linear alkyl group containing 10 to 18 carbon atoms, Ph is a phenyl group and X is an alkali metal and/or alkaline earth metal, ammonium, alkylammonium, alkanolammonium or glucammonium. Dodecyl benzenesulfonates, tetradecyl benzenesulfonates, hexadecyl benzenesulfonates and technical mixtures thereof in the form of the sodium salts are preferably used. Alkyl and/or alkenyl sulfates, which are also often referred to as fatty alcohol sulfates, are understood to be the sulfation products of primary alcohols which preferably correspond to formula R″O—SO₃X, in which R″ is a linear or branched, aliphatic alkyl and/or alkenyl group containing 6 to 22 and preferably 12 to 18 carbon atoms and X is an alkali metal and/or alkaline earth metal, ammonium, alkylammonium, alkanolammonium or glucammonium. Typical examples of alkyl sulfates which may be used in accordance with the invention are the sulfation products of caproic alcohol, caprylic alcohol, capric alcohol, 2-ethylhexyl alcohol, lauryl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol and erucyl alcohol and the technical mixtures thereof obtained by high-pressure hydrogenation of technical methyl ester fractions or aldehydes from Roelen's oxosynthesis. The sulfation products may advantageously be used in the form of their alkali metal salts, more especially their sodium salts. Alkyl sulfates based on C_{16/18} tallow fatty alcohols or vegetable fatty alcohols with a comparable C-chain distribution in the form of their sodium salts are particularly preferred. Alkyl ether sulfates may also be used in combination with chitosans or chitosan derivatives. Alkyl ether sulfates (“ether sulfates”) are known anionic surfactants which, on an industrial scale, are produced by SO₃ or chlorosulfonic acid (CSA) sulfation of fatty alcohol or oxoalcohol polyglycol ethers and subsequent neutralization. Ether sulfates suitable for use in accordance with the invention correspond to formula R′″O—(CH₂CH₂O), in which R′″ is a linear or branched alkyl and/or alkenyl group containing 6 to 22 carbon atoms, n is a number of 1 to 10 and X is an alkali metal and/or alkaline earth metal, ammonium, alkylammonium, alkanolammonium or glucammonium. Typical examples are the sulfates of addition products of on average 1 to 10 and more particularly 2 to 5 mol ethylene oxide onto caproic alcohol, caprylic alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol and technical mixtures thereof in the form of their sodium and/or magnesium salts. The ether sulfates may have both a conventional homolog distribution and a narrow homolog distribution. It is particularly preferred to use ether sulfates based on adducts of on average 2 to 3 mol ethylene oxide with technical C_{12/14} or C_{12/18} coconut fatty alcohol fractions in the form of their sodium and/or magnesium salts.

The use of chitosans with the combination of monoglyceride ether sulfates and fatty acid isethionate, fatty acid taurate and/or fatty acid sarcosinate condensation products in accordance with U.S. Pat. No. 6,013,616 is excluded from the statement of claim.

The aqueous preparations preferably have a neutral pH but, more particularly, an acidic pH. Values of 4.0 to 7.0 are typical. The pH is preferably adjusted to a value of 5.0 to 6.5.

The aqueous preparations according to the invention, which may contain chitosan and other suitable ingredients as described above, may advantageously be used as so-called protective fluids. Behind this is the idea that the user first brings his/her skin into contact with the protective fluid before dishwashing, the chitosan or chitosan derivative forming a protective film on the skin so that the user can then bring the skin thus protected into contact with the dishwashing detergent or the water-containing dishwashing liquor. Such protective fluids contain the chitosan or chitosan derivative in quantities of 0.01 to 5% by weight, preferably in quantities of 0.05 to 2.5% by weight and more particularly in quantities of 0.1 to 2.5% by weight, based on the preparation as a whole.

Ethanol may also be used, for example in quantities of 1 to 45% by weight, preferably in quantities of 5 to 35% by weight and more particularly in quantities of 5 to 8% by weight.

The aqueous preparations may also contain other film-forming compounds, such as for example acrylic acid copolymers, cellulose derivatives, vinyl pyrrolidone/vinyl acetate copolymers in various quantity ratios, polymers based on vinyl pyrrolidone/vinyl acetate and vinyl propionate, polyethylene oxide resins, polyvinyl acetate, polyvinyl alcohol and protein hydrolyzates. Film formers based on natural resins are decolored shellac, sandarak resin, benzoin resins and rosin. Semisynthetic products (condensation products of rosin and acrylic acid) are also suitable. Film formers are understood to be substances of different composition which, after dissolution in a solvent (water, ethanol or others), are applied to or sprayed onto the skin or hair and, after evaporation of the solvent, form films which, besides protective and sealing functions, can also perform supporting functions. These additional film formers may be present in the aqueous preparations according to the invention in quantities of 1 to 35% by weight, preferably in quantities of 1 to 25% by weight and more particularly in quantities of 1 to 10% by weight.

Another preferred, but optional constituent in the aqueous chitosan-containing preparations according to the invention are proteins or protein derivatives, more particularly protein hydrolyzates. Protein hydrolyzates are degradation products of animal or vegetable proteins, for example collagen, elastin or keratin and, preferably, almond and potato protein and, more particularly, silk, wheat, rice and soya protein, which are hydrolyzed by acidic, alkaline and/or enzymatic hydrolysis and, thereafter, have a molecular weight in the range from 100 to 500,000 and preferably in the range from 100 to 50,000. Other preferred molecular weight ranges are 500 to 5,000 and, more particularly, 600 to 4,000. Although protein hydrolyzates, in the absence of a hydrophobic residue, are not surfactants in the accepted sense, they are often used for the formulation of surface-active compositions by virtue of their dispersing properties.

In addition, the aqueous preparations according to the invention may contain other ingredients, particularly those which protect or care for the human skin. Such ingredients include, for example, plant extracts (aqueous, alcohol and/or ether extracts). However, the preparations may also contain other cosmetically compatible waxes and polymers, vitamins, plant-based active principles, for example aloe vera, and UV filters, preservatives, perfumes oils and fragrances, consistency factors, solubilizers, thickeners, hydrotropes, emulsifiers, pearlizers and dyes. The other ingredients may be introduced into the formulations in the form of liposomes or so-called sponges.

A typical formulation for such preparations contains 0.01 to at most 5% by weight chitosan or chitosan derivatives, 1 to 5% by weight amphoteric surfactants and 1 to 5% by weight nonionic and/or cationic surfactants and 0.01 to 2% by weight preservatives. The pH is preferably adjusted to a value of 4 to 8. The balance to 100% by weight is water, demineralized water being particularly preferred in every case. If a foaming nonionic surfactant is used, these general formulations are suitable for the production of protective foams for the skin in accordance with the present technical teaching. These preparations have a composition similar to that of the protective fluids described above, although they still contain a foaming surfactant, the alkyl oligoglycoside compounds and/or cationic surfactants mentioned above being preferred.

The following general formulation is suitable as a protective fluid: 0.1 to 5% by weight chitosan or chitosan derivative, 0.1 to 5% by weight of a skin-care component or mixture of skin-care components (for example protein hydrolyzates) and 7 to 30% by weight ethanol. The balance to 100% by weight is again (preferably demineralized) water. The quantities mentioned are based on active substance.

The present invention also relates to a skin-friendly manual dishwashing process in which the skin of the hands is first contacted with a chitosan-containing preparation, after which the dishes are manually cleaned in known manner with a dishwashing detergent.

The present teaching also encompasses the notion of incorporating chitosan or chitosan derivatives in known and typical dishwashing detergent formulations. Such formulations typically contain 10 to 30% by weight anionic surfactants, 1 to 10% by weight amphoteric surfactants and 0 to 10% by weight nonionic and/or cationic surfactants. The chitosan derivatives may preferably even be used in dishwashing detergents which contain anionic surfactants.

### EXAMPLES

The following aqueous preparations were produced (Table 1—all quantities in % by weight active substance).

| | 1 | 2 | 3 | 4 | 5 | 6 |
|---|---|---|---|---|---|---|
| Chitosan¹⁾ | 0.75 | 0.75 | 0.75 | 0.75 | 0.75 | 0.5 |
| Wheat protein hydrolyzate²⁾ | | 1 | | | | 1 |
| N,N-Dimethyl-N-(cocoamido- | 1.5 | 1.5 | | 1.2 | | |
| propyl)-ammonium acetobetaine | | | | | | |
| C₈₋₁₀ Alkyl polyglucoside | | | 1.2 | 0.6 | | |
| C₈₋₁₆ Alkyl-1,4-glucoside | | | | | 2.5 | |
| Preservative | 0.1 | 0.1 | 0.1 | 0.1 | 0.1 | |
| Ethanol | | | | | | 20 |
| Water | to 100 | to 100 | to 100 | to 100 | to 100 | to 100 |

¹⁾Hydagen ® HCMS-LA (Cognis, 50,000 to 1,000,000 g/mol, degree of deacetylation min. 80%)
²⁾Gluadin ® W 40 (Cognis)

Formulations 1 to 5 may be used in foam form as a so-called protection mousse. Formulation 6 is a liquid which is applied to the hands, for example before manual dishwashing.

What is claimed is:

1. A detergent composition having improved dermatological compatibility, comprising
(A) a manual dishwashing detergent mixture and
(B) a chitosan preparation selected from a chitosan, a chitosan derivative and a mixture thereof having a molecular weight of 50,000 to 1,200,000 or a molecular weight of 300,000 to 2,000,000, wherein the detergent composition is substantially free of monoglyceride ether sulfates or of fatty acid isethionate, fatty acid taurate and fatty acid sarcosinate condensation products.

2. The detergent composition of claim 1 wherein the chitosan preparation has a molecular weight of 50,000 to 1,200,000 and a Brookfield viscosity of 5,000 mPas.

3. The detergent composition of claim 1 wherein the chitosan preparation is an aqueous chitosan preparation having a pH of 4 to 8.

4. The detergent composition of claim 3 wherein the aqueous chitosan preparation contains 0.01 to 2.5% by weight of a chitosan, a chitosan derivative or a mixture thereof.

5. The detergent composition of claim 3 wherein the aqueous chitosan preparation contains 0.05 to 1.0% by weight of a chitosan, a chitosan derivative or a mixture thereof.

6. The detergent composition of claim 3 wherein the aqueous chitosan preparation is substantially free from anionic surfactants.

7. The detergent composition of claim 3 wherein the aqueous chitosan preparation is substantially free from cationic quaternary ammonium compounds containing ester groups.

8. The detergent composition of claim 3 wherein the aqueous chitosan preparation is substantially free from esterquats.

9. The detergent composition of claim 1 wherein the chitosan preparation is in the form of a foam.

10. The detergent composition of claim 3 wherein the aqueous chitosan preparation further contains a nonaqueous solvent.

11. The detergent composition of claim 10 wherein the nonaqueous solvent is ethanol.

12. The detergent composition of claim 3 wherein the aqueous chitosan preparation further contains an alkyl (oligo)glycoside.

13. The detergent composition of claim 13 wherein the alkyl (oligo)glycoside is an alkyl or alkenyl oligoglucoside.

14. An aqueous dermatological composition for protecting the skin from deleterious effects of detergent compositions, comprising 0.01 to 5% by weight, based on the composition as a whole, of a chitosan preparation selected from a chitosan, a chitosan derivative and a mixture thereof, and water.

15. The aqueous dermatological composition of claim 14 wherein the chitosan preparation has a molecular weight of 50,000 to 1,200,000 and a Brookfield viscosity of 5,000 mPas.

16. The aqueous dermatological composition of claim 14 further comprising ethanol.

17. The aqueous dermatological composition of claim 14 wherein the water is demineralized water.

18. A process for improving the dermatological compatibility of a detergent mixture, comprising combining
(A) a manual dishwashing detergent mixture and
(B) a chitosan preparation selected from a chitosan, a chitosan derivative and a mixture thereof having a molecular weight of 50,000 to 1,200,000 or a molecular weight of 300,000 to 2,000,000, wherein the detergent composition is substantially free of monoglyceride ether sulfates or of fatty acid isethionate, fatty acid taurate and fatty acid sarcosinate condensation product.

19. The process claim 18 wherein the chitosan preparation has a molecular weight of 50,000 to 1,200,000 and a Brookfield viscosity of 5,000 mPas.

20. The process of claim 18 wherein the chitosan preparation is an aqueous chitosan preparation having a pH of 4 to 8.

21. The process of claim 18 wherein the aqueous chitosan preparation contains 0.01 to 2.5% by weight of a chitosan, a chitosan derivative or a mixture thereof.

22. The process of claim 18 wherein the aqueous chitosan preparation is substantially free from anionic surfactants.
